(19) United States
(12) Patent Application Publication
(10) Pub. No.: US 2013/0017544 A1
(43) Pub. Date: Jan. 17, 2013

(54) High Resolution Melting Analysis on a Droplet Actuator

(71) Applicants: Allen E. Eckhardt, Durham, NC (US); Zhishan Hua, Greensboro, NC (US)

(72) Inventors: Allen E. Eckhardt, Durham, NC (US); Zhishan Hua, Greensboro, NC (US)

(73) Assignee: Advanced Liquid Logic Inc, Research Triangle Park, NC (US)

(21) Appl. No.: 13/546,817

(22) Filed: Jul. 11, 2012

Related U.S. Application Data
(60) Provisional application No. 61/506,358, filed on Jul. 11, 2011.

(51) Int. Cl. G01N 25/04; C12M 1/33; C12M 1/34; C12Q 1/68; G01N 21/64

(52) U.S. Cl. 435/6.11; 435/6.12; 435/287.2; 435/306.1

(57) ABSTRACT

An integrated droplet actuator device and methods are provided for performing PCR amplification and high-resolution melting (HRM) analysis on a single droplet actuator. HRM analysis can be used in combination with PCR amplification for detection of sequence variations (e.g., single-nucleotide polymorphisms, nucleotide-repeat polymorphisms, mutation scanning and assessment of DNA methylation) within one or more genes of interest. The PCR amplicons can be fluorescently labeled during amplification using a saturating DNA intercalating fluorescent dye, a 5′-labeled primer, or labeled probes. Also provided are a droplet actuator device and methods for sample preparation using the droplet actuator and detection of sequence variations on the same droplet actuator.

### 1 CROSS REFERENCE TO RELATED APPLICATIONS

This application claims the benefit of U.S. provisional application No. 61/506,358 filed Jul. 11, 2011, the disclosure of which is hereby incorporated by reference in its entirety.

### 2 TECHNICAL FIELD

The present disclosure relates to methods for high resolution melting analysis on a droplet actuator.

### 3 BACKGROUND

A droplet actuator typically includes one or more substrates configured to form a surface or gap for conducting droplet operations. The one or more substrates establish a droplet operations surface or gap for conducting droplet operations and may also include electrodes arrange to conduct the droplet operations. The droplet operations substrate or the gap between the substrates may be coated or filled with a filler fluid that is immiscible with the liquid that forms the droplets.

Droplet actuators are used to conduct genetic analysis using polymerase chain reaction (PCR) technologies. In one application, PCR may be used in combination with other molecular techniques used to detect sequence variations within a gene of interest. In one example, PCR may be used in combination with high-resolution melting (HRM) analysis for detection of sequence variations within a gene of interest. HRM analysis is used to characterize DNA samples according to their dissociation behavior as they transition from double stranded DNA to single stranded DNA with increasing temperature. DNA samples may be characterized based on sequence length, GC content and DNA sequence complimentarity. PCR in combination with HRM analysis may, for example, be used to detect single-nucleotide polymorphisms, nucleotide-repeat polymorphisms, and assessment of DNA methylation. The PCR amplicons may be fluorescently labeled during amplification using a saturating DNA intercalating dye, a 5′-labeled primer, or labeled probes. There is a need for techniques that make use of a droplet actuator for combined amplification and HRM analysis for detection of sequence variations within a gene of interest.

### 4 SUMMARY

This Summary is provided to introduce a selection of concepts in a simplified form that are further described below in the Description.

Systems and methods for high resolution melting analysis on a droplet actuator are disclosed herein. According to one or more embodiments, a method is provided for integrating PCR amplification and high-resolution melting (HRM) analysis, the method including using a droplet actuator for: positioning a sample droplet comprising a target DNA template for amplification within a first temperature control zone such that the target DNA template is single stranded; merging the sample droplet with a reagent droplet comprising PCR primers, a label allowing for detection of the target DNA template, dNTPs, buffers, and DNA polymerase to yield a reaction droplet; transporting the reaction droplet to a second temperature control zone and incubating the reaction droplet for primer annealing and extension; transporting the reaction droplet between the first and second temperature control zones for a number of amplification cycles of the target DNA template; after amplification, heating and cooling the reaction droplet for heteroduplex formation of the amplified target DNA for discrimination of alleles; and performing a HRM analysis on the amplified target DNA. The method can include detecting one or more of sequence variations, polymorphisms, mutations, or methylation within the amplified target DNA. The target DNA template for amplification can be FRM1 associated with Fragile X syndrome, the PCR primers can be selected to amplify a region of the CGG repeat domain of the FRM1 gene for discrimination of alleles, and the HRM analysis can correlate a FRM1 melting point with a length of the region of the CGG repeat domain for detection of Fragile X syndrome.

According to one or more embodiments, a method is provided for preparing genomic DNA from a biological sample, the method including using a droplet actuator for: receiving a biological sample comprising cells into a well that contains fluid, such that the cells are released into the fluid; lysing the cells such that the genomic DNA is released into the fluid; recovering the DNA such that the DNA is bound to a bead suspended within a droplet; and washing the DNA-bound beads within the droplet to remove unbound material such that the genomic DNA is prepared. The biological sample can be a buccal swab. The method can include eluting the DNA from the DNA-bound beads such that the DNA is contained in the droplet surrounding the beads and transporting the droplet containing the DNA away from the beads for further processing of the DNA using the droplet actuator. The genomic DNA can be FRM1 associated with Fragile X syndrome, and the further processing can be HRM analysis to determine a FRM1 melting profile for detection of Fragile X syndrome.

According to one or more embodiments, a system is provided for preparing genomic DNA from a biological sample, the system including a droplet actuator comprising a well and configured to: receive a biological sample including cells into the well that contains fluid, such that the cells are released into the fluid; lyse the cells such that the genomic DNA is released into the fluid; recover the DNA such that the DNA is bound to a bead suspended within the fluid; and wash the DNA-bound beads within a droplet of the fluid to remove unbound material such that the genomic DNA is prepared. The droplet actuator can include a first substrate configured to define the well; a second substrate defining an opening that provides a pathway between the well and a gap, wherein the gap is defined by the second substrate and a third substrate; and a dispensing electrode substantially aligned with the opening and integrated with the third substrate for performing droplet operations in the gap. The droplet actuator can include droplet operations electrodes integrated with the third substrate for performing droplet operations in the gap. The droplet actuator can include a magnet arranged in close proximity to one of the droplet operations electrodes for washing the DNA-bound beads.

### 5 DEFINITIONS

As used herein, the following terms have the meanings indicated.

“Activate,” with reference to one or more electrodes, means affecting a change in the electrical state of the one or more electrodes which, in the presence of a droplet, results in a droplet operation. Activation of an electrode can be accomplished using alternating or direct current. Any suitable voltage may be used. For example, an electrode may be activated using a voltage which is greater than about 150 V, or greater than about 200 V, or greater than about 250 V, or from about 275 V to about 375 V, or about 300 V. Where alternating current is used, any suitable frequency may be employed. For example, an electrode may be activated using alternating current having a frequency from about 1 Hz to about 100 Hz, or from about 10 Hz to about 60 Hz, or from about 20 Hz to about 40 Hz, or about 30 Hz.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical, amorphous and other three dimensional shapes. The bead may, for example, be capable of being subjected to a droplet operation in a droplet on a droplet actuator or otherwise configured with respect to a droplet actuator in a manner which permits a droplet on the droplet actuator to be brought into contact with the bead on the droplet actuator and/or off the droplet actuator. Beads may be provided in a droplet, in a droplet operations gap, or on a droplet operations surface. Beads may be provided in a reservoir that is external to a droplet operations gap or situated apart from a droplet operations surface, and the reservoir may be associated with a fluid path that permits a droplet including the beads to be brought into a droplet operations gap or into contact with a droplet operations surface. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead, a portion of a bead, or only one component of a bead. The remainder of the bead may include, among other things, polymeric material, coatings, and moieties which permit attachment of an assay reagent. Examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication Nos. 20050260686, entitled “Multiplex flow assays preferably with magnetic particles as solid phase,” published on Nov. 24, 2005; 20030132538, entitled “Encapsulation of discrete quanta of fluorescent particles,” published on Jul. 17, 2003; 20050118574, entitled “Multiplexed Analysis of Clinical Specimens Apparatus and Method,” published on Jun. 2, 2005; 20050277197. Entitled “Microparticles with Multiple Fluorescent Signals and Methods of Using Same,” published on Dec. 15, 2005; 20060159962, entitled “Magnetic Microspheres for use in Fluorescence-based Applications,” published on Jul. 20, 2006; the entire disclosures of which are incorporated herein by reference for their teaching concerning beads and magnetically responsive materials and beads. Beads may be pre-coupled with a biomolecule or other substance that is able to bind to and form a complex with a biomolecule. Beads may be pre-coupled with an antibody, protein or antigen, DNA/RNA probe or any other molecule with an affinity for a desired target. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference. Bead characteristics may be employed in the multiplexing aspects of the invention. Examples of beads having characteristics suitable for multiplexing, as well as methods of detecting and analyzing signals emitted from such beads, may be found in U.S. Patent Publication No. 20080305481, entitled “Systems and Methods for Multiplex Analysis of PCR in Real Time,” published on Dec. 11, 2008; U.S. Patent Publication No. 20080151240, “Methods and Systems for Dynamic Range Expansion,” published on Jun. 26, 2008; U.S. Patent Publication No. 20070207513, entitled “Methods, Products, and Kits for Identifying an Analyte in a Sample,” published on Sep. 6, 2007; U.S. Patent Publication No. 20070064990, entitled “Methods and Systems for Image Data Processing,” published on Mar. 22, 2007; U.S. Patent Publication No. 20060159962, entitled “Magnetic Microspheres for use in Fluorescence-based Applications,” published on Jul. 20, 2006; U.S. Patent Publication No. 20050277197, entitled “Microparticles with Multiple Fluorescent Signals and Methods of Using Same,” published on Dec. 15, 2005; and U.S. Patent Publication No. 20050118574, entitled “Multiplexed Analysis of Clinical Specimens Apparatus and Method,” published on Jun. 2, 2005.

“Droplet” means a volume of liquid on a droplet actuator. Typically, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. As yet another example, a droplet may be bounded by filler fluid and the atmosphere. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on filed on Jan. 30, 2006; Pollack et al., International Patent Application No. PCT/US2006/047486, entitled “Droplet-Based Biochemistry,” filed on Dec. 11, 2006; Shenderov, U.S. Pat. Nos. 6,773,566, entitled “Electrostatic Actuators for Microfluidics and Methods for Using Same,” issued on Aug. 10, 2004 and 6,565,727, entitled “Actuators for Microfluidics Without Moving Parts,” issued on Jan. 24, 2000; Kim and/or Shah et al., U.S. patent application Ser. Nos. 10/343,261, entitled “Electrowetting-driven Micropumping,” filed on Jan. 27, 2003, 11/275,668, entitled “Method and Apparatus for Promoting the Complete Transfer of Liquid Drops from a Nozzle,” filed on Jan. 23, 2006, 11/460,188, entitled “Small Object Moving on Printed Circuit Board,” filed on Jan. 23, 2006, 12/465,935, entitled “Method for Using Magnetic Particles in Droplet Microfluidics,” filed on May 14, 2009, and 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. Nos. 7,641,779, entitled “Method and Apparatus for Programmable fluidic Processing,” issued on Jan. 5, 2010, and 6,977,033, entitled “Method and Apparatus for Programmable fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels: Electronic Liquid Transport with Continuous Channel Functionality,” Lab Chip, 10:832-836 (2010); the entire disclosures of which are incorporated herein by reference, along with their priority documents. Certain droplet actuators will include one or more substrates arranged with a gap therebetween and electrodes associated with (e.g., layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface. A top substrate may also be provided, which is separated from the droplet operations surface by a gap, commonly referred to as a droplet operations gap. Various electrode arrangements on the top and/or bottom substrates are discussed in the above-referenced patents and applications and certain novel electrode arrangements are discussed in the description of the invention. During droplet operations it is preferred that droplets remain in continuous contact or frequent contact with a ground or reference electrode. A ground or reference electrode may be associated with the top substrate facing the gap, the bottom substrate facing the gap, in the gap. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. In one embodiment, a conductive material (e.g., an epoxy, such as MASTER BOND™ Polymer System EP79, available from Master Bond, Inc., Hackensack, N.J.) provides the electrical connection between electrodes on one substrate and electrical paths on the other substrates, e.g., a ground electrode on a top substrate may be coupled to an electrical path on a bottom substrate by such a conductive material. Where multiple substrates are used, a spacer may be provided between the substrates to determine the height of the gap therebetween and define dispensing reservoirs. The spacer height may, for example, be from about 5 μm to about 600 μm, or about 100 μm to about 400 μm, or about 200 μm to about 350 μm, or about 250 μm to about 300 μm, or about 275 μm. The spacer may, for example, be formed of a layer of projections form the top or bottom substrates, and/or a material inserted between the top and bottom substrates. One or more openings may be provided in the one or more substrates for forming a fluid path through which liquid may be delivered into the droplet operations gap. The one or more openings may in some cases be aligned for interaction with one or more electrodes, e.g., aligned such that liquid flowed through the opening will come into sufficient proximity with one or more droplet operations electrodes to permit a droplet operation to be effected by the droplet operations electrodes using the liquid. The base (or bottom) and top substrates may in some cases be formed as one integral component. One or more reference electrodes may be provided on the base (or bottom) and/or top substrates and/or in the gap. Examples of reference electrode arrangements are provided in the above referenced patents and patent applications. In various embodiments, the manipulation of droplets by a droplet actuator may be electrode mediated, e.g., electrowetting mediated or dielectrophoresis mediated or Coulombic force mediated. Examples of other techniques for controlling droplet operations that may be used in the droplet actuators of the invention include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed to conduct a droplet operation in a droplet actuator of the invention. Similarly, one or more of the foregoing may be used to deliver liquid into a droplet operations gap, e.g., from a reservoir in another device or from an external reservoir of the droplet actuator (e.g., a reservoir associated with a droplet actuator substrate and a fluid path from the reservoir into the droplet operations gap). Droplet operations surfaces of certain droplet actuators of the invention may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and NOVEC™ electronic coatings (available from 3M Company, St. Paul, Minn.), and other fluorinated monomers for plasma-enhanced chemical vapor deposition (PECVD). In some cases, the droplet operations surface may include a hydrophobic coating having a thickness ranging from about 10 nm to about 1,000 nm. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene) poly(styrenesulfonate) (PEDOT:PSS). Other examples of electrically conducting organic polymers and alternative conductive layers are described in Pollack et al., International Patent Application No. PCT/US2010/040705, entitled “Droplet Actuator Devices and Methods,” the entire disclosure of which is incorporated herein by reference. One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness in the range of about 20 to about 200 nm, preferably about 50 to about 150 nm, or about 75 to about 125 nm, or about 100 nm. In some cases, the top and/or bottom substrate includes a PCB substrate that is coated with a dielectric, such as a polyimide dielectric, which may in some cases also be coated or otherwise treated to make the droplet operations surface hydrophobic. When the substrate includes a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.).; NELCO® N4000-6 and N5000-30/32 (available from Park Electrochemical Corp., Melville, N.Y.); ISOLA™ FR406 (available from Isola Group, Chandler, Ariz.), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT®nonwoven aramid reinforcement (available from DuPont, Wilmington, Del.); NOMEX® brand fiber (available from DuPont, Wilmington, Del.); and paper. Various materials are also suitable for use as the dielectric component of the substrate. Examples include: vapor deposited dielectric, such as PARYLENE™ C (especially on glass) and PARYLENE™ N (available from Parylene Coating Services, Inc., Katy, Tex.); TEFLON® AF coatings; cytop; soldermasks, such as liquid photoimageable soldermasks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film soldermask, such as those in the VACREL® dry film soldermask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; and polypropylene. Droplet transport voltage and frequency may be selected for performance with reagents used in specific assay protocols. Design parameters may be varied, e.g., number and placement of on-chip reservoirs, number of independent electrode connections, size (volume) of different reservoirs, placement of magnets/bead washing zones, electrode size, inter-electrode pitch, and gap height (between top and bottom substrates) may be varied for use with specific reagents, protocols, droplet volumes, etc. In some cases, a substrate of the invention may derivatized with low surface-energy materials or chemistries, e.g., using deposition or in situ synthesis using poly- or per-fluorinated compounds in solution or polymerizable monomers. Examples include TEFLON® AF coatings and FLUOROPEL® coatings for dip or spray coating, and other fluorinated monomers for plasma-enhanced chemical vapor deposition (PECVD). Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray industries, Inc., Japan. Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution. An example of reconstitutable reagents suitable for use with the invention includes those described in Meathrel, et al., U.S. Pat. No. 7,727,466, entitled “Disintegratable films for diagnostic devices,” granted on Jun. 1, 2010.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.” Impedance or capacitance sensing or imaging techniques may sometimes be used to determine or confirm the outcome of a droplet operation. Examples of such techniques are described in Sturmer et al., International Patent Pub. No. WO/2008/101194, entitled “Capacitance Detection in a Droplet Actuator,” published on Aug. 21, 2008, the entire disclosure of which is incorporated herein by reference. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may exceed about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to electrowetting area; in other words, 1×-, 2×-3×-droplets are usefully controlled operated using 1, 2, and 3 electrodes, respectively. If the droplet footprint is greater than the number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

“Filler fluid” means a fluid associated with a droplet operations substrate of a droplet actuator, which fluid is sufficiently immiscible with a droplet phase to render the droplet phase subject to electrode-mediated droplet operations. For example, the gap of a droplet actuator is typically filled with a filler fluid. The filler fluid may, for example, be a low-viscosity oil, such as silicone oil or hexadecane filler fluid. The filler fluid may fill the entire gap of the droplet actuator or may coat one or more surfaces of the droplet actuator. Filler fluids may be conductive or non-conductive. Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents used in the specific assay protocols and effective interaction or non-interaction with droplet actuator materials. Examples of filler fluids and filler fluid formulations suitable for use with the invention are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated herein by reference, as well as the other patents and patent applications cited herein.

“Immobilize” with respect to magnetically responsive beads, means that the beads are substantially restrained in position in a droplet or in filler fluid on a droplet actuator. For example, in one embodiment, immobilized beads are sufficiently restrained in position in a droplet to permit execution of a droplet splitting operation, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP.

“Transporting into the magnetic field of a magnet,” “transporting towards a magnet,” and the like, as used herein to refer to droplets and/or magnetically responsive beads within droplets, is intended to refer to transporting into a region of a magnetic field capable of substantially attracting magnetically responsive beads in the droplet. Similarly, “transporting away from a magnet or magnetic field,” “transporting out of the magnetic field of a magnet,” and the like, as used herein to refer to droplets and/or magnetically responsive beads within droplets, is intended to refer to transporting away from a region of a magnetic field capable of substantially attracting magnetically responsive beads in the droplet, whether or not the droplet or magnetically responsive beads is completely removed from the magnetic field. It will be appreciated that in any of such cases described herein, the droplet may be transported towards or away from the desired region of the magnetic field, and/or the desired region of the magnetic field may be moved towards or away from the droplet. Reference to an electrode, a droplet, or magnetically responsive beads being “within” or “in” a magnetic field, or the like, is intended to describe a situation in which the electrode is situated in a manner which permits the electrode to transport a droplet into and/or away from a desired region of a magnetic field, or the droplet or magnetically responsive beads is/are situated in a desired region of the magnetic field, in each case where the magnetic field in the desired region is capable of substantially attracting any magnetically responsive beads in the droplet. Similarly, reference to an electrode, a droplet, or magnetically responsive beads being “outside of” or “away from” a magnetic field, and the like, is intended to describe a situation in which the electrode is situated in a manner which permits the electrode to transport a droplet away from a certain region of a magnetic field, or the droplet or magnetically responsive beads is/are situated away from a certain region of the magnetic field, in each case where the magnetic field in such region is not capable of substantially attracting any magnetically responsive beads in the droplet or in which any remaining attraction does not eliminate the effectiveness of droplet operations conducted in the region. In various aspects of the invention, a system, a droplet actuator, or another component of a system may include a magnet, such as one or more permanent magnets (e.g., a single cylindrical or bar magnet or an array of such magnets, such as a Halbach array) or an electromagnet or array of electromagnets, to form a magnetic field for interacting with magnetically responsive beads or other components on chip. Such interactions may, for example, include substantially immobilizing or restraining movement or flow of magnetically responsive beads during storage or in a droplet during a droplet operation or pulling magnetically responsive beads out of a droplet.

“Washing” with respect to washing a bead means reducing the amount and/or concentration of one or more substances in contact with the bead or exposed to the bead from a droplet in contact with the bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### 6 BRIEF DESCRIPTION OF THE DRAWINGS

FIGS. 1A through 1E illustrate top views of an example of a portion of an electrode arrangement 100 of a droplet actuator and show a process of integrating PCR amplification and HRM analysis for allele discrimination on a droplet actuator; and

FIGS. 2A and 2B illustrate side views of a portion of a droplet actuator 200 and show a process of integrating sample preparation from a buccal swab on a droplet actuator.

### 7 DESCRIPTION

The present invention provides an integrated droplet actuator device and methods for performing PCR amplification and high-resolution melting (HRM) analysis on a single droplet actuator. HRM analysis may be used in combination with PCR amplification for detection of sequence variations (e.g., single-nucleotide polymorphisms, nucleotide-repeat polymorphisms, mutation scanning and assessment of DNA methylation) within one or more genes of interest. The PCR amplicons may be fluorescently labeled during amplification using a saturating DNA intercalating fluorescent dye, a 5′-labeled primer, or labeled probes. In various embodiments, the invention also provides for droplet actuator-based sample preparation and detection of sequence variations on the same droplet actuator.

The integrated droplet actuator device of the invention is designed to fit onto the deck of an instrument that is configured to provide precise temperature adjustments and sensitive fluorescence resolution for PCR amplification and high-resolution melting discrimination of sequence variations.

In one embodiment, the droplet actuator device and methods of the invention may be used for preparation of genomic DNA, target PCR amplification and HRM analysis for genotyping Fragile X syndrome.

### 7.1 Integrated Digital Microfluidic PCR and HRM

The digital microfluidic protocol for detection of sequence variations (e.g., polymorphisms, mutations, and methylation) within a gene of interest combines PCR amplification of target sequences and high-resolution melting (HRM) analysis of the target amplicons on a single droplet actuator. HRM analysis is based on the physical property of DNA melting temperature for a double-stranded target sequence (i.e., amplicon) of a gene of interest. Each gene in an organism (individual) is typically present in two (or more) copies, i.e., two alleles. The alleles may be the same, i.e., homozygous, or different, i.e., heterozygous. During amplification of a DNA sample, both alleles are amplified. As the amplified DNA is denatured and cooled post-PCR for HRM analysis, different combinations of annealed double-stranded amplicons may be formed. Homozygous samples result in the formation of homoduplexes. Due to differences in sequence composition, different homozygous samples have different denaturation temperatures that result in different melt curves. Heterozygous samples contain two different alleles, which result in the formation of both homoduplexes (i.e., two homoduplex products) and heteroduplexes (i.e., two heteroduplex products). Heteroduplexes arise from the annealing of non-complementary strands of DNA, which form, for example, during fast cooling of the sample. Because of the mis-paired regions in the heteroduplexes, the double-stranded amplicon is less stable and therefore dissociates at a lower temperature. The lower melting temperature produces a different melt curve profile. Because a different melt curve profile is produced, heterozygous samples may be differentiated from homozygous samples.

In the digital microfluidic protocol, rapid PCR thermocycling may be performed in a flow-through format where for each cycle the reaction droplets are cyclically transported between different temperature zones within the oil filled droplet actuator. Incorporation of a fluorescent label in the target amplicons may be used to monitor the PCR reaction and for subsequent HRM analysis. In one embodiment, target amplicons may be fluorescently labeled during PCR amplification using a saturating double-stranded DNA intercalating dye such as LCGreen (available from Idaho Technology Inc, Salt Lake City, Utah), EvaGreen (available from Biotium Inc, Hayward, Calif.), or SYTO 9 (available from Invitrogen™ by Life Technologies Corp, Carlsbad, Calif.). In another embodiment, a 5′-fluorescently labeled primer may be used to label the target amplicons. In yet another embodiment, fluorescently labeled probes may be used to label the target amplicons. Established PCR protocols that include optimum cycling parameters and concentration of reagents including Taq polymerase, buffers and primers (forward and reverse primers) may be selected for each gene of interest. For example, the sequence and length of the forward and reverse primers may be selected to produce amplicons of sufficient length for precise discrimination of alleles. The concentration of each primer, primer annealing temperature and magnesium concentration may be selected to provide specific amplification of the gene of interest with high yield. Annealing/extension time and number of thermocycles may be selected to provide high quality amplicons and rapid throughput in a PCR-HRM integrated protocol.

Established HRM protocols for allele discrimination may be adapted for use on a droplet actuator^{1, 2}. For example, prior to HRM analysis, the amplified DNA is typically subjected to a final round of denaturation and annealing selected to enhance heteroduplex formation. The rate of denaturation and cooling may be selected for substantial formation of heteroduplexes. In one example, a higher heating rate (e.g., 0.4° C./second) and a rapid cooling rate (e.g., about >0.1° C./second to about <5° C./seconds) may be selected to produce a higher number of heteroduplexes for more accurate discrimination of alleles. In another example, the ionic strength (e.g., a lower ionic strength) of the annealing buffer may be selected for substantial formation of heteroduplexes. Final HRM analysis may be performed on the duplexed DNA amplicons using direct melting, i.e., precise warming of the DNA amplicons from about 50° C. to about 95° C. at a selected temperature transition rate (e.g., 0.05° C./second).

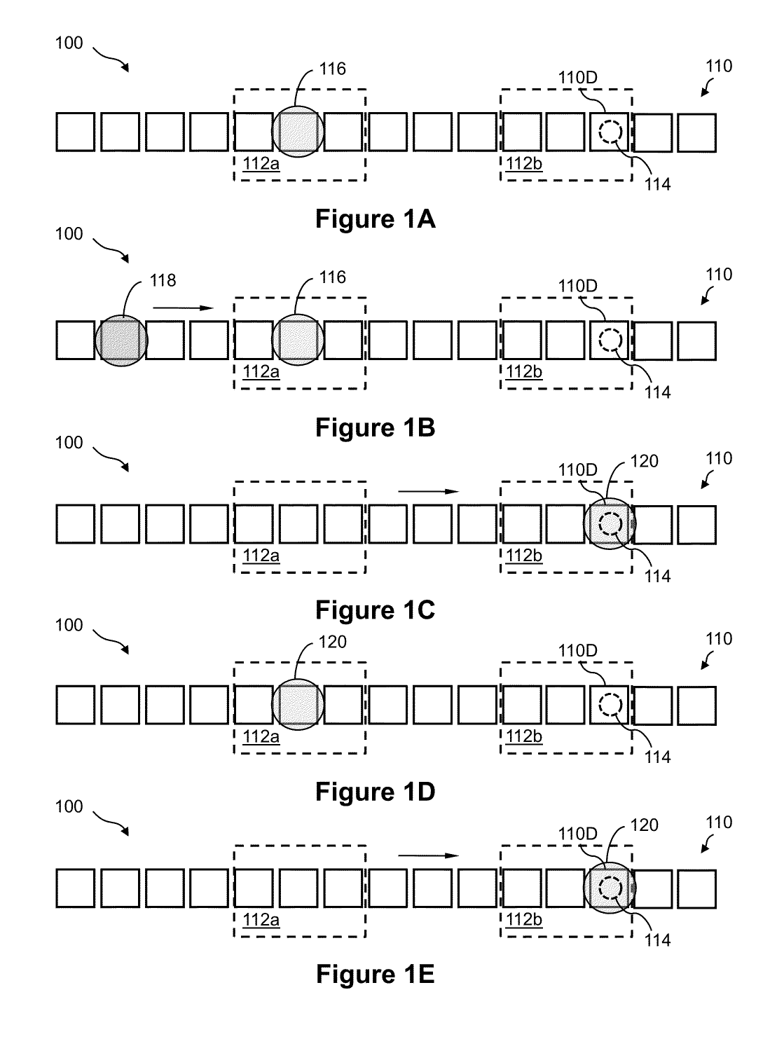

FIGS. 1A through 1E illustrate top views of an example of a portion of an electrode arrangement 100 of a droplet actuator and show a process of integrating PCR amplification and HRM analysis for allele discrimination on a droplet actuator. In this example, the droplet actuator is used for integrating PCR amplification and high-resolution melting (HRM) analysis. The method of the invention of FIGS. 1A through 1E is an example of an amplification and HRM analysis protocol wherein target amplicons may be fluorescently labeled during PCR amplification using a saturating double-stranded DNA intercalating dye such as LCGreen. Intercalating dyes bind specifically to double-stranded DNA. When the intercalating dye is bound to double-stranded DNA, a fluorescent signal is produced. During HRM analysis, as the double-stranded DNA is heated and the two strands of the DNA melt apart, the presence of double stranded DNA decreases and consequently the fluorescence signal is reduced. The rate of fluorescence decrease is generally greatest near the melting temperature (Tₘ) of the PCR product. The melting temperature is a function of PCR product characteristics, including GC-content (Tₘ is higher in GC-rich PCR products), length, and sequence content. The data may be acquired and plotted as a melt curve showing relative fluorescence versus temperature and/or derived melting peaks.

Electrode arrangement 100 may include an arrangement of droplet operations electrodes 110 that is configured for PCR amplification and HRM analysis. Droplet operations are conducted atop droplet operations electrodes 110 on a droplet operations surface. Two temperature control zones 112, such as temperature control zone 112a and 112b, may be associated with electrode arrangement 100. Thermal control elements (not shown) control the temperature of filler fluid (not shown) in the vicinity of temperature control zones 112a and 112b. For example, temperature control zone 112a may be heated to about 95° C., which is a temperature sufficient for denaturation of double-stranded DNA. Temperature control zone 112b may, for example, be heated to about 55° C., which is a temperature sufficient for primer annealing and extension. In one example, temperature control zones 112a and 112b may be used for PCR thermocycling. In another example, thermal conditions in temperature control zone 112b may be adjusted for acquisition of a melting curve for HRM analysis. While two temperature control zones 112 are shown, any number of temperature control zones 112 may be associated with electrode arrangement 110. A detection spot 114 may be arranged in close proximity to droplet operations electrode 110D within temperature control zone 112b.

An example of a general process of PCR amplification and HRM analysis may include, but is not limited to, the following steps.

In one step, FIG. 1A shows a sample droplet 116 that is positioned at a certain droplet operations electrode 110 within temperature control zone 112a. Sample droplet 116 may, for example, include nucleic acid template (genomic DNA target) for amplification. In one example, the nucleic acid template may include a variant region of interest for a particular gene. Because sample droplet 116 is within temperature control zone 112a, the nucleic acid template is denatured (single-stranded).

In other steps, FIGS. 1B and 1C show an incubation process in which a reagent droplet 118 is merged using droplet operations with sample droplet 116 within temperature control zone 112a to yield a reaction droplet 120. Reagent droplet 118 may include primers and PCR reagents (e.g., dNTPs, buffers, DNA polymerase) for target amplification. Reagent droplet 118 may also include a fluorescent saturating DNA intercalating dye such as LCGreen. Reaction droplet 120 is transported using droplet operations to a certain droplet operations electrode 110 within temperature control zone 112b. Reaction droplet 120 is incubated in temperature control zone 112b for a period of time that is sufficient for primer annealing/extension and incorporation of the fluorescent intercalating dye. Reaction droplet 120 may be repeatedly transported back and forth for any number of cycles using droplet operations between thermal reaction zones 112b and 112a for PCR amplification of target DNA.

Referring to FIG. 1C, reaction droplet 120 may be transported using droplet operations to droplet operations electrode 110D, which is within the range of detection spot 114. An imaging device (e.g., fluorimeter, not shown), arranged in proximity of detection spot 114, is used to capture and quantitate the amount of fluorescence in reaction droplet 120. The term “detector” is herein used interchangeably, for the purposes of the specification, drawings, and claims, with the term “imaging device”. A detector can be positioned in the proximity of the second temperature control zone to detect the labeled target DNA template. Amplified nucleic acid may be detected after any number of amplification cycles (i.e., real-time or end-point).

In another step, FIG. 1D shows reaction droplet 120 transported, after completion of PCR amplification, using droplet operations to a certain droplet operations electrode 110 within temperature control zone 112a. In this step, a final denaturation and cooling of the amplified DNA within reaction droplet 120 is performed to produce a high number of heteroduplexes for more accurate discrimination of alleles. In one example, the temperature within temperature control zone 112a may be adjusted to provide a higher heating rate (e.g., 0.4° C./second) and a rapid cooling rate (e.g., about >0.1° C./second to about <5° C./seconds) that enhances heteroduplex formation.

In another step, FIG. 1E shows reaction droplet 120 transported using droplet operations to a droplet operations electrode 110D within temperature control zone 112b, which is within the range of detection spot 114. In this step, HRM analysis is performed. In one example, the temperature within temperature control zone 112b may be adjusted at a ramping rate of 0.2° C./second from about 50° C. to about 95° C. An imaging device (e.g., fluorimeter, not shown), arranged in proximity of detection spot 114, is used to continuously capture and quantitate the amount of fluorescence in reaction droplet 120 as the temperature is increased.

### 7.2 Preparation of Genomic DNA on a Droplet Actuator

Genomic DNA from a biological sample may be prepared on a droplet actuator. In one example, genomic DNA, such as genomic DNA from cells obtained from a buccal swab (i.e., cells from the cheek), may be prepared using, for example, Dynabeads DNA DIRECT (available from Invitrogen™ by Life Technologies Corp, Carlsbad, Calif.). In this example, genomic DNA may be isolated on a droplet actuator directly from the buccal swab. In another example, genomic DNA may be prepared using ChargeSwitch magnetic beads (available from Invitrogen™ by Life Technologies Corp, Carlsbad, Calif.).

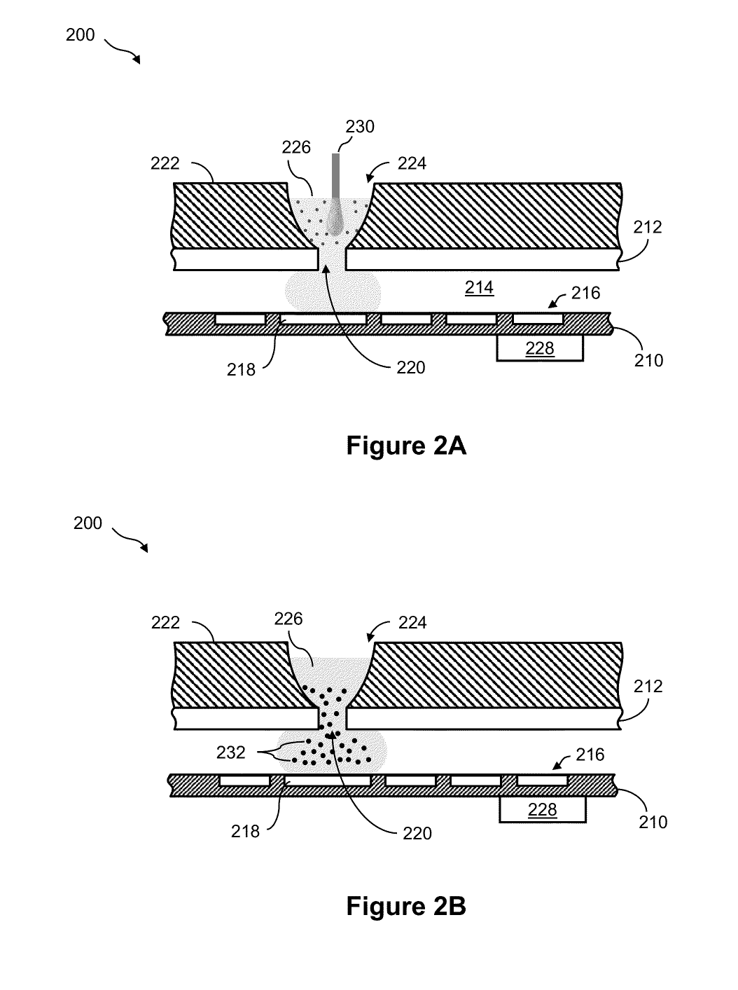

FIGS. 2A and 2B illustrate side views of a portion of a droplet actuator 200 and show a process of integrating sample preparation from a buccal swab on a droplet actuator. Droplet actuator 200 may include a bottom substrate 210 that is separated from a top substrate 212 by a gap 214. An arrangement of droplet operations electrodes 216 (e.g., electrowetting electrodes) and a dispensing electrode 218 may be disposed on bottom substrate 210. Droplet operations are conducted atop droplet operations electrodes 216 on a droplet operations surface. An opening 220 may be provided within top substrate 212. Opening 220 is substantially aligned with dispensing electrode 218. A substrate 222 may be disposed atop top substrate 212. Substrate 222 may include a well 224, which is suitable for delivering liquid through opening 220 and into gap 214. Well 224 contains a quantity of fluid 226. Fluid 226 may, for example, be a lysis solution. A magnet 228 is arranged in close proximity to droplet operations electrodes 216. For example, magnet 228 is arranged such that a certain droplet operations electrodes 216 (e.g., droplet operations electrode 216) is within the magnetic field thereof. Magnet 228 may, for example, be a permanent magnet or an electromagnet.

An example of a process of preparing a DNA sample from a biological sample, such as a buccal swab may include, but is not limited to, the following steps.

In one step, FIG. 2A shows a sample collection and lysis protocol in which a swab 230 is used to collect a sample, such as a cell sample from the cheek of a subject. Swab 230 is then placed into well 224 that contains fluid 226 in order to resuspend the sample and release the cells into the solution. One or more different lysing reagents may be added to fluid 226 and incubated at one or more different temperatures to yield a lysed cell solution that contains released DNA. In a specific example, swab 230 is incubated in 200 μL of fluid 126 (0.05 M sodium hydroxide) and heated at 95° C. for 10 minutes.

In another step, FIG. 2B shows a DNA recovery process in which a quantity of magnetically responsive beads 232, such as Dynabeads, is added to the lysed cell solution. The lysed cell solution with magnetically responsive beads 232 therein is incubated for a sufficient period of time for released DNA to bind beads 232. The bead-containing lysed cell solution is then loaded onto dispensing electrode 218. One or more DNA capture droplets (not shown) may be transported using droplet operations into the presence of magnet 228 and washed using a merge-and-split wash protocol to remove unbound material, yielding a washed bead-containing droplet substantially lacking in unbound material (not shown). In one embodiment, the purified DNA is then eluted from beads 232 with 10 mM Tris HCl, 1 mM EDTA, pH 7.4. The eluted DNA contained in the droplet surrounding the Dynabeads may then be transported away from the Dynabeads for further processing on the droplet actuator, e.g., for execution of a droplet-based integrated PCR amplification and HRM assay.

In another embodiment, genomic DNA may be may be prepared using ChargeSwitch magnetic beads. One or more DNA capture droplets (not shown) may be transported to a temperature control zone (not shown) and the purified genomic DNA may, for example, be denatured by alkali treatment (NaOH) at 42° C. The magnetic beads with bound genomic DNA thereon may be dispensed for further processing on the droplet actuator, e.g., for execution of methylation profiling by melting curve analysis.

### 7.3 Example Application: Genotyping Fragile X Syndrome

The invention provides integrated PCR amplification and HRM analysis methods for detection of Fragile X syndrome on a droplet actuator. Fragile X syndrome is associated with the expansion of a single CGG trinucleotide repeat in the 5′-untranslated region of the fragile X-mental retardation 1 (FMR1) gene on the X chromosome. The FMR1 protein encoded by this gene is required for normal neural development. Among people without the fragile X mutation, the number of CGG repeats varies from 6 to about 40. The fragile X mutation involves an expanded number of the CGG repeats. Expansions from about 55 to about 200 CGG repeats, called permutations, are seen in unaffected carriers. About 40 to about 55 repeats is considered a “grey zone” where normal and permutation size ranges overlap. Expansions with more than 200 repeats, called full mutations, are associated with increased methylation of that region of the DNA which effectively silences the expression of the FMR1 protein.

In one embodiment, the invention provides methods for a droplet-based integrated PCR amplification and HRM assay that correlates FRM1 amplicon melting point with the length of the CGG repeat domain. The melting temperature of a DNA molecule is dependent on both the length of the molecule and the specific nucleotide sequence composition of that molecule (e.g., a higher Tₘ is associated with a higher GC content). In one example, the PCR primers may be selected to amplify a region of the CGG repeat domain of the FRM1 alleles which have been shown to be associated with Fragile X syndrome. Primer pairs (forward and reverse primers) may be selected to produce amplicons of sufficient length for precise discrimination of alleles within the polymorphic CGG region. PCR amplification and HRM analysis may be performed as described in reference to FIG. 1.

In another embodiment, the invention provides methods for a droplet-based integrated PCR amplification and HRM assay that correlates FRM1 amplicon melting point with methylation of the FRM1 allele. Existing assays for fragile X syndrome based on detection of hypermethylated FMR1 alleles by methylation-specific melting curve analysis may be adapted for use on a droplet actuator³. In general, methylation-specific melting curve analysis uses sodium bisulfite treatment of isolated genomic DNA prior to PCR amplification. Bisulfite treatment is used to convert unmethylated cytosines to uracil, while methylated cytosines remain unchanged. The uracil is then converted to thymine during subsequent PCR amplification, while the methylcytosine will be amplified as cytosine. PCR products generated from bisulfate-treated DNA templates with different contents of methylcytosine show differences in melting temperature, which may be resolved by melting analysis. The melting profiles may be used to differentiate among four different methylation states: unmethylated alleles generate a single low melting peak, fully methylated alleles generate a single high melting peak, a mixture of unmethylated and fully methylated alleles generate both the low and high melting peaks, and heterogeneously methylated alleles generate a broadened melting top located between the low and high melting peaks³.

In one example, single-tube analysis of DNA methylation using silica superparamagnetic beads (SSBs)⁵ may be adapted for use on a droplet actuator. An example of a digital microfluidic protocol for methylation-specific melting curve analysis may include, but is not limited to, the following: Genomic DNA may be prepared on a droplet actuator from a buccal swab, as described in reference to FIGS. 2A and 2B, using superparamagnetic beads such as ChargeSwitch beads. A sample droplet that includes magnetic beads with purified genomic DNA thereon is dispensed and transported using droplet operations to a temperature control zone and the purified genomic DNA is denatured using, for example, alkali treatment (NaOH) at 42° C. The sample droplet with denatured DNA therein is combined using droplet operations with a bisulfite reagent droplet to yield a reaction droplet. The reaction droplet is incubated, for example, at 55° C. for a period of time sufficient for conversion of unmethylated cytosines to uracil. Following bisulfite conversion, the reaction droplet is transported using droplet operations into the presence of a magnet and washed using a merge-and-split wash protocol to purify the converted DNA. The purified DNA is then eluted from the ChargeSwitch beads with 10 mM Tris HCl, 1 mM EDTA, pH 7.4. The eluted DNA contained in the droplet surrounding the ChargeSwitch beads may then be transported away from the beads for execution of a droplet-based integrated PCR amplification and HRM analysis. PCR amplification and HRM analysis may be performed on the converted sample droplet as described in reference to FIG. 1. For PCR amplification, primers may be selected for methylation-insensitive amplification or methylation-sensitive amplification.

### 7.4 Systems

It will be appreciated that various aspects of the invention may be embodied as a method, system, computer readable medium, and/or computer program product. Aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Program code for carrying out operations of the invention may be written in an object oriented programming language such as Java, Smalltalk, C++ or the like. However, the program code for carrying out operations of the invention may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user's computer, for example, may entirely execute the program code or only partly execute the program code. The program code may be a stand-alone software package that is at least partly on the user's computer and/or partly executed on a remote computer or entirely on a remote computer or server. In the latter scenario, the remote computer may be connected to the user's computer through a communications network.

The invention may be applied regardless of networking environment. The communications network may be a cable network operating in the radio-frequency domain and/or the Internet Protocol (IP) domain. The communications network, however, may also include a distributed computing network, such as the Internet (sometimes alternatively known as the “World Wide Web”), an intranet, a local-area network (LAN), and/or a wide-area network (WAN). The communications network may include coaxial cables, copper wires, fiber optic lines, and/or hybrid-coaxial lines. The communications network may even include wireless portions utilizing any portion of the electromagnetic spectrum and any signaling standard (such as the IEEE 802 family of standards, GSM/CDMA/TDMA or any cellular standard, and/or the ISM band). The communications network may even include powerline portions, in which signals are communicated via electrical wiring. The invention may be applied to any wireless/wireline communications network, regardless of physical componentry, physical configuration, or communications standard(s).

Certain aspects of invention are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the invention.

### REFERENCES

1 C. T. Wittwer, G. H. Reed, C. N. Gundry, J. G. Vandersteen, and R. J. Pryor. High-Resolution Genotyping by Amplicon Melting Analysis Using LCGreen. Clinical Chemistry 49 (6): 853-860, 2003.
2 C. N. Gundry, J. G. Vandersteen, G. H. Reed, R. J. Pryor, J. Chen, and C. T. Wittwer. Amplicon Melting Analysis with Labeled Primers: A Closed-Tube Method for Differentiating Homozygotes and Heterozygotes. Clinical Chemistry 49 (3): 396-406, 2003.
3 C. Dahl, K. Gronskov, L. A. Larsen, P. Guldberg, and K. Brondum-Nielsen. A homogeneous assay for analysis of FRM1 promoter methylation in patients with fragile X syndrome. Clinical Chemistry 53 (4): 790-793, 2007.
4 J. Worm, A. Aggerholm, and P. Guldberg. In-Tube DNA Methylation Profiling by Fluorescence Melting Curve Analysis. Clinical Chemistry 47 (7): 1183-1189, 2001.
5 V. J. Bailey, Y. Zhang, B. P. Keeley, C. Yin, K. L. Pelosky, M. Brock, S. B. Baylin, J. G. Herman, and T. Wang. Single-Tube Analysis of DNA Methylation Using Silica Superparamagnetic Beads. Clinical Chemistry 56 (6): 1022-1025, 2010.

### 9 CONCLUDING REMARKS

The foregoing detailed description of embodiments refers to the accompanying drawings, which illustrate specific embodiments of the invention. Other embodiments having different structures and operations do not depart from the scope of the present invention. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

What is claimed:

1. A method for integrating PCR amplification and high-resolution melting (HRM) analysis, the method comprising:
using a droplet actuator for:
positioning a sample droplet comprising a target DNA template for amplification within a first temperature control zone such that the target DNA template is single stranded;
merging the sample droplet with a reagent droplet comprising PCR primers, a label allowing for detection of the target DNA template, dNTPs, buffers, and DNA polymerase to yield a reaction droplet;
transporting the reaction droplet to a second temperature control zone and incubating the reaction droplet for primer annealing and extension;
transporting the reaction droplet between the first and second temperature control zones for a number of amplification cycles of the target DNA template;
after amplification, heating and cooling the reaction droplet for heteroduplex formation of the amplified target DNA for discrimination of alleles; and
performing a HRM analysis on the amplified target DNA.

2. The method of claim 1, wherein the label comprises a saturating DNA intercalating dye, a 5′-labeled primer, or a labeled probe.

3. The method of claim 1, wherein the label is a fluorescent label.

4. The method of claim 1, comprising detecting the amplified target DNA after the number of amplification cycles.

5. The method of claim 4, wherein the number of amplification cycles is real-time or end-point.

6. The method of claim 1, comprising using a detector positioned in the proximity of the second temperature control zone to detect the labeled target DNA.

7. The method of claim 6, wherein the label is a fluorescent saturating DNA intercalating dye,
wherein the method comprises using the detector to capture and quantitate the amount of fluorescence in the reaction droplet in the target DNA, and
wherein the number of amplification cycles for fluorescence capture is real-time or end-point.

8. The method of claim 6, wherein the label is a fluorescent saturating DNA intercalating dye, and
wherein the method comprises using the detector to continuously capture and quantitate the amount of fluorescence in the reaction droplet in the HRM analysis.

9. The method of claim 1, wherein performing the HRM analysis comprises adjusting temperature at a ramping rate of 0.2° C./second from about 50° C. to about 95° C.

10. The method of claim 1, comprising detecting one or more of sequence variations, polymorphisms, mutations, or methylation within the amplified target DNA.

11. The method of claim 1, wherein the target DNA template for amplification is FRM1 associated with Fragile X syndrome,
wherein the PCR primers are selected to amplify a region of the CGG repeat domain of the FRM1 gene for discrimination of alleles, and
wherein the HRM analysis correlates a FRM1 melting point with a length of the region of the CGG repeat domain for detection of Fragile X syndrome.

12. The method of claim 1, wherein the target DNA template for amplification is FRM1 associated with Fragile X syndrome in which unmethylated cytosines have been converted to uracil,
wherein the PCR primers are selected to amplify a region of the CGG repeat domain of the FRM1 gene for discrimination of alleles, and
wherein the HRM analysis is a methylation-specific melting curve analysis for detection of Fragile X syndrome.

13. A system for integrating PCR amplification and high-resolution melting (HRM) analysis, the system comprising:
a droplet actuator configured to:
position a sample droplet comprising a target DNA template for amplification within a first temperature control zone such that the target DNA template is single stranded;
merge the sample droplet with a reagent droplet comprising PCR primers, a label allowing for detection of the target DNA template, dNTPs, buffers, and DNA polymerase to yield a reaction droplet;
transport the reaction droplet to a second temperature control zone and incubate the reaction droplet for primer annealing and extension;
transport the reaction droplet between the first and second temperature control zones for a number of amplification cycles of the target DNA template;
after amplification, heat and cool the reaction droplet for heteroduplex formation of the amplified target DNA for discrimination of alleles; and
perform a HRM analysis on the amplified target DNA.

14. The system of claim 13, wherein the label comprises a saturating DNA intercalating dye, a 5′-labeled primer, or a labeled probe.

15. The system of claim 13, wherein the label is a fluorescent label.

16. The system of claim 13, wherein the droplet actuator is configured to detect the amplified target DNA after the number of amplification cycles.

17. The system of claim 16, wherein the number of amplification cycles is real-time or end-point.

18. The system of claim 13, wherein the droplet actuator comprises a detector positioned in the proximity of the second temperature control zone to detect the labeled target DNA.

19. The system of claim 18, wherein the label is a fluorescent saturating DNA intercalating dye,
wherein the detector is configured to capture and quantitate the amount of fluorescence in the reaction droplet in the target DNA, and
wherein the number of amplification cycles for fluorescence capture is real-time or end-point.

20. The system of claim 18, wherein the label is a fluorescent saturating DNA intercalating dye, and
wherein the detector is configured to continuously capture and quantitate the amount of fluorescence in the reaction droplet in the HRM analysis.

21. The system of claim 13, wherein the droplet actuator is configured to adjust HRM analysis temperature at a ramping rate of 0.2° C./second from about 50° C. to about 95° C.

22. The system of claim 13, wherein the droplet actuator is configured to detect one or more of sequence variations, polymorphisms, mutations, or methylation within the amplified target DNA.

23. The system of claim 13, wherein the target DNA template for amplification is FRM1 associated with Fragile X syndrome,
wherein the PCR primers are selected to amplify a region of the CGG repeat domain of the FRM1 gene for discrimination of alleles, and
wherein the HRM analysis correlates a FRM1 melting point with a length of the region of the CGG repeat domain for detection of Fragile X syndrome.

24. The system of claim 13, wherein the target DNA template for amplification is FRM1 associated with Fragile X syndrome in which unmethylated cytosines have been converted to uracil,
wherein the PCR primers are selected to amplify a region of the CGG repeat domain of the FRM1 gene for discrimination of alleles, and
wherein the HRM analysis is a methylation-specific melting curve analysis for detection of Fragile X syndrome.

25. A method for preparing genomic DNA from a biological sample, the method comprising:
using a droplet actuator for:
receiving a biological sample comprising cells into a well that contains fluid, such that the cells are released into the fluid;
lysing the cells such that the genomic DNA is released into the fluid;
recovering the DNA such that the DNA is bound to a bead suspended within a droplet; and
washing the DNA-bound beads within the droplet to remove unbound material such that the genomic DNA is prepared.

26. The method of claim 25, wherein lysing the cells comprises adding one or more lysing reagents to the fluid and incubating at one or more temperatures.

27. The method of claim 25, wherein the beads are magnetically responsive beads, and
wherein washing the DNA-bound beads comprises using a merge-and-split wash protocol with the droplet being in the presence of a magnet.

28. The method of claim 25, further comprising dispensing the droplet for further processing of the droplet using the droplet actuator.

29. The method of claim 25, further comprising:
eluting the DNA from the DNA-bound beads such that the DNA is contained in the droplet surrounding the beads; and
transporting the droplet containing the DNA away from the beads for further processing of the DNA using the droplet actuator.

30. The method of claim 25, wherein the biological sample is a buccal swab.

31. The method of claim 25, further comprising:
denaturing the prepared bead-bound genomic DNA within the droplet;
combining the droplet with a bisulfite comprising reagent droplet to yield a reaction droplet;
incubating the reaction droplet at a temperature and for a time period sufficient for conversion of unmethylated cytosines to uracil; and
washing the bead-bound genomic DNA.

32. The method of claim 31, wherein the genomic DNA is FRM1 associated with Fragile X syndrome, and
wherein the conversion of unmethylated cytosines to uracil allows for use of an HRM analysis to determine a FRM1 melting profile for detection of Fragile X syndrome.

33. The method of claim 31, wherein the genomic DNA is FRM1 associated with Fragile X syndrome, and
wherein the method further comprises:
eluting the DNA from the DNA-bound beads such that the DNA is contained in the droplet surrounding the beads; and
transporting the droplet containing the DNA away from the beads for HRM analysis using the droplet actuator to determine a FRM1 melting profile for detection of Fragile X syndrome.

34. A system for preparing genomic DNA from a biological sample, the system comprising:
a droplet actuator comprising a well and configured to:
receive a biological sample comprising cells into the well that contains fluid, such that the cells are released into the fluid;
lyse the cells such that the genomic DNA is released into the fluid;
recover the DNA such that the DNA is bound to a bead suspended within the fluid; and
wash the DNA-bound beads within a droplet of the fluid to remove unbound material such that the genomic DNA is prepared.

35. The system of claim 34, wherein the droplet actuator is configured to add one or more lysing reagents to the fluid and incubate at one or more temperatures.

36. The system of claim 34, wherein the beads are magnetically responsive beads, and
wherein the droplet actuator is configured to use a merge-and-split wash protocol with the droplet being in the presence of a magnet.

37. The system of claim 34, wherein the droplet actuator is configured to dispense the droplet for further processing using the droplet actuator.

38. The system of claim 34, wherein the droplet actuator is configured to:
elute the DNA from the DNA-bound beads such that the DNA is contained in the droplet surrounding the beads; and
transport the droplet containing the DNA away from the beads for further processing of the DNA using the droplet actuator.

39. The system of claim 34, wherein the biological sample is a buccal swab.

40. The system of claim 34, wherein the droplet actuator is configured to:
denature the prepared bead-bound genomic DNA within the droplet;
combine the droplet with a bisulfite comprising reagent droplet to yield a reaction droplet;
incubate the reaction droplet at a temperature and for a time period sufficient for conversion of unmethylated cytosines to uracil; and
wash the bead-bound genomic DNA within the reaction droplet.

41. The system of claim 40, wherein the genomic DNA is FRM1 associated with Fragile X syndrome, and
wherein the conversion of unmethylated cytosines to uracil allows for use of an HRM analysis to determine a FRM1 melting profile for detection of Fragile X syndrome.

42. The system of claim 40, wherein the genomic DNA is FRM1 associated with Fragile X syndrome, and
wherein the droplet actuator is configured to:
elute the DNA from the DNA-bound beads such that the DNA is contained in the droplet surrounding the beads; and
transport the droplet containing the DNA away from the beads for HRM analysis using the droplet actuator to determine a FRM1 melting profile for detection of Fragile X syndrome.

43. The system of claim 34, wherein the droplet actuator comprises:
a first substrate configured to define the well;
a second substrate defining an opening that provides a pathway between the well and a gap, wherein the gap is defined by the second substrate and a third substrate; and
a dispensing electrode substantially aligned with the opening and integrated with the third substrate for performing droplet operations in the gap.

44. The system of claim 43, comprising droplet operations electrodes integrated with the third substrate for performing droplet operations in the gap.

45. The system of claim 44, comprising a magnet arranged in close proximity to one of the droplet operations electrodes for washing the DNA-bound beads.
